(19) United States
(12) Patent Application Publication
(10) Pub. No.: US 2021/0000588 A1
(43) Pub. Date: Jan. 7, 2021

(54) 3-D PRINTED TITANIUM POROUS BIOTENODESIS SCREW WITH SUTURE ANCHOR

(71) Applicant: Jarrett D. Cain, Hummelstown, PA (US)

(72) Inventor: Jarrett D. Cain, Hummelstown, PA (US)

(21) Appl. No.: 16/920,032

(22) Filed: Jul. 2, 2020

Related U.S. Application Data
(60) Provisional application No. 62/869,940, filed on Jul. 2, 2019.

(51) Int. Cl. A61F 2/08; A61B 17/04

(52) U.S. Cl. CPC A61F 2/0811; A61B 17/0401; A61F 2002/0852; A61B 2017/044; A61B 2017/0464

(57) ABSTRACT

A 3-D printed titanium porous screw adapted for placement within a bone tunnel, where the screw comprises a top section with a plurality of step-tapered edges, a middle section with porous inner lattice structure, and a lower section with tip portion for insertion into a bone tunnel. The plurality of step-tapered edges increases insertion torque and provides enhanced bone-to-implant contact and improved bone incorporation. The porous inner lattice structure allows the ingrowth of a patient's own bone quality to lessen the chance of rejection and loss of strength. Manufacturing of the 3-D printed lattice bone suture may be based on pre-operative MRI and CT scans that may be transferred to a software program to determine the integrity of the tendon as well as the evaluation of the bones involved. This allows the screw with suture anchor to be patient-specific in terms of the lattice formation and placement of the screw.

### FIELD OF INVENTION

The present invention relates to the field of screws and suture anchors used in surgical operations, and more particularly to titanium screws with patient-specific 3-D printed porous inner lattice bone suture anchors utilized to attach tendons, ligaments and other soft tissue to bone.

### BACKGROUND

Biocompatible screws with suture anchors for attaching tendons, ligaments and other soft tissue to bone are known in the art. Currently on the market, the suture anchors and Biotenodesis screws utilize Poly L Lactic Acid (PLLA) and other composites that can lead to delayed reaction and loosening of fixation. Therefore, with the current suture anchors and screws, the objectives of bone-tendon fixation, increasing failure load, and maintaining close contact between the screw and subsequent bone growth are compromised due to the PLLA and other composites that cause loosening of fixation and reaction delay. Accordingly, there is a need for a screw with suture anchor that may allow for incorporation of bone growth without loss of fixation. It would be advantageous if the surgical screw could also be supplemented with additional functionality to be patient-specific in terms of placement of the suture anchor as well as a lattice formation that may allow the ingrowth of the patient's own bone quality to lessen the chance of rejection and loss of strength.

### SUMMARY

There is provided according to the embodiments of the invention a surgical screw comprising a top section having a head surface comprising a loop member for attachment of a suture to the screw, a plurality of substantially planar ledges each having a step-tapered edge, and an angle disposed between each of the substantially planar ledges and the respective step-tapered edges. Further, the surgical screw also comprises a middle section having a 3-D printed porous inner lattice structure, and a lower section including a point configured for insertion into a bone tunnel. The middle section is disposed between the top section and the lower section, and the 3-D printed porous inner lattice structure is dependent upon a patient's bone, joint and tendon attributes as determined by pre-operative surgical planning with radiographs, MRI, and CT scans.

In another aspect, the present disclosure is directed to a surgical screw comprising a top section having a head surface comprising a loop member for attachment of a suture to the screw, a middle section having a 3-D printed porous inner lattice structure, and a lower section including a point configured for insertion into a bone tunnel. The middle section is disposed between the top section and the lower section, and the 3-D printed porous inner lattice structure is dependent upon a patient's bone, joint, and tendon attributes as determined by pre-operative surgical planning with radiographs, MRI, and CT scans.

In another aspect, the present disclosure is directed to a surgical screw comprising a top section having a head surface comprising a loop member for attachment of a suture to the screw, and a middle section having a 3-D printed porous inner lattice structure. The 3-D printed porous inner lattice structure of the middle section is comprised of a cross-sectional structure with a plurality of spaces between the cross-sectional structure. The diameter of the 3-D printed porous inner lattice structure is approximately 3 millimeters, and the 3-D printed porous inner lattice structure is dependent upon a patient's bone, joint, and tendon attributes as determined by pre-operative surgical planning with radiographs, MRI, and CT scans.

These and other objects, features and advantages will be apparent from the following detailed description of illustrative embodiments thereof, which is to be read in connection with the accompanying drawings.

### BRIEF DESCRIPTION OF THE DRAWINGS

A more detailed understanding may be had from the following description, given by way of example in conjunction with the accompanying drawings herein.

FIG. 1A shows a perspective view of a titanium screw, with a top section having a plurality of step-tapered edges and planar ledges, a middle section with a 3-D printed porous inner lattice structure, and a lower section with tip portion for insertion into a bone tunnel.

FIG. 1B shows a top perspective view of the titanium screw, illustrating the top section having a plurality of step-tapered edges and planar ledges, and the middle section with a 3-D printed porous inner lattice structure.

FIG. 1C shows a perspective view of a titanium screw, illustrating the functionality of the screw at the top section, with a FiberWire suture attached to the loop member.

FIG. 2 shows a cross-sectional view of a titanium screw, with a top section having a plurality of step-tapered edges and planar ledges, a middle section with a 3-D printed porous inner lattice structure, and a lower section with tip portion for insertion into a bone tunnel.

FIG. 3 shows an additional cross-sectional view of a titanium screw, with a top section having a plurality of step-tapered edges and planar ledges, a middle section with a 3-D printed porous inner lattice structure, and a lower section with tip portion for insertion into a bone tunnel.

FIG. 4A shows a cross-sectional view of a top section of the titanium screw, with a plurality of step-tapered edges and planar ledges.

FIG. 4B shows an additional cross-sectional view of a top section of the titanium screw, with the angles between the respective step-tapered edges and planar ledges.

FIG. 5 shows an axial view providing the diameters of the outer titanium surface of the screw and of the 3-D printed porous inner lattice structure.

FIG. 6 shows an additional axial view of the outer titanium surface of the screw and the 3-D printed porous inner lattice structure.

### DETAILED DESCRIPTION OF THE PREFERRED EMBODIMENTS

Various embodiments are described herein where like references to figures are used to describe like features. Each feature or element may be used alone without other features and elements or in various combinations with or without other features and elements.

The present embodiments relate to a titanium screw with 3-D printed porous inner lattice bone suture anchor for complex foot and ankle surgery. In general, Biotenodesis screws and suture anchors are utilized for attaching tendons, ligaments and other soft tissue to bone. The titanium screw with 3-D printed porous inner lattice bone suture anchor discussed herein provides for the incorporation of bone into the lattice formation of the middle section of the screw to reduce the chance of rejection of previously used PLLA material or decomposition of bio-absorbable material that would lose strength of tendon repair and transfer. The middle section with inner lattice structure allows for bone ingrowth, for incorporation of bone into the suture anchor, without utilization of the PLLA material or decomposition of bio-absorbable material that can lead to delayed reaction and loosening of fixation. In addition, the surgical screw with suture anchor in the embodiments described herein also provides additional patient-specific functionality, based upon 3-D manufacturing, to accommodate the particular anatomical attributes of a given patient's bone and soft tissue geometry.

Manufacturing of the 3-D printed porous inner lattice bone suture anchor may be based on pre-operative radiographs, MRI and CT scans (non-weight bearing vs. weight bearing). The scanned information may be transferred to a software program that may determine the integrity of the tendon involved in the transfer as well as the evaluation of the bones involved. This may allow the screw with suture anchor to be patient-specific to improve patient outcomes by recognizing the individual patient's anatomical geometry of the bone and tissue. Using 3-D printing, a patient-specific screw with suture anchor may be manufactured with titanium porous structure and lattice bone suture anchor formation. In particular, the patient-specific, 3-D printed porous inner lattice bone suture anchor formation may allow for the incorporation of bone into the suture anchor. The dimensions and geometry of the 3-D printed lattice bone suture anchor formation may be tailored to the specific characteristics of the patient's bone and soft tissue structure. Such 3-D manufacturing may provide a patient-specific screw with suture anchor customized for the degree of the patient's ailment or injury and projected recovery time, in relation to the patient's age and medical history, for example.

In general, a procedure utilizing the proposed screw involves insertion of a guide pin, which may vary based on the size of the screw. Once the guide pin is placed, a reamer is drilled over the guide pin to a designated black line that will not allow the bone tunnel to be over-drilled to the point that the suture anchor is too deep. Following the creation of the bone tunnel, the proper bone anchor and pilot hole diameter depend on size of the tendon. Bone tunnel depth should generally be two millimeters longer than the length of the suture anchor. Once the suture anchor is placed, a number 2 FiberWire suture is used to create a stitch around the bone to allow the appropriate tendon to bone insertion. The size of the screw may generally be dependent upon analysis of the bone, joint, and tendon by pre-operative surgical planning with radiographs, MRI, and CT scans.

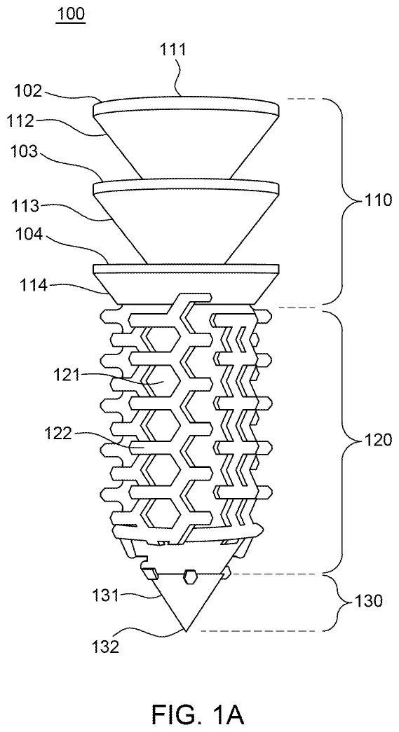

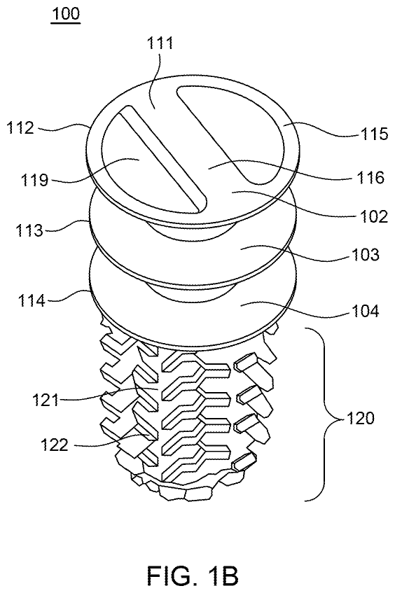

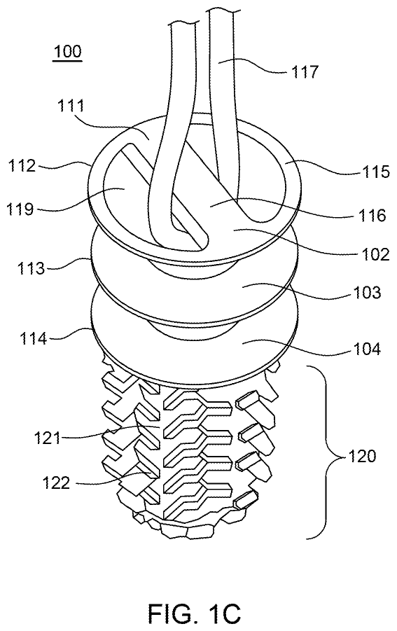

FIG. 1A shows an example embodiment of the 3-D printed titanium porous screw with suture anchor 100. The screw with suture anchor 100 shown in FIG. 1 is oriented vertically. Based upon its 3-D manufacturing, the screw 100 of FIG. 1A may be supplemented with additional functionality to be patient-specific in terms of placement of the screw with suture anchor 100, as well as manufacturing of the 3-D printed lattice structure based on pre-operative MRI and CT scans that may allow the ingrowth of the patient's own bone quality to lessen the chance of rejection and loss of strength.

Referring again to FIG. 1A, the screw 100 may feature a top section 110 including a plurality of substantially planar ledges 102, 103, 104 extending laterally and perpendicularly to the axis of the screw 100. Angled, step-tapered edges 112, 113, 114, respectively meet each ledge 102, 103, 104 at an angle, which may generally be less than 90 degrees to provide a screw thread in the top section 110. The angled, step-tapered edges 112, 113, 114 may each provide for enhanced bone-to-implant contact to improve soft tissue and bone fixation. The screw 100 may also feature a middle section 120 comprising a 3-D printed lattice bone suture anchor with a porous inner lattice structure 121, 122, and a lower section 130 including an angled edge 131 leading to a point 132 for insertion into a bone tunnel. The screw 100 may be composed of titanium or other suitable metals or metallic alloys. Although the embodiment of FIG. 1A shows three substantially planar ledges 102, 103, 104 and three step-tapered edges 112, 113, 114, it is understood that in other embodiments of the screw 100, additional ledges and step-tapered edges may be featured in the screw 100, or fewer ledges and step-tapered edges may be featured in the screw 100.

The middle section 120 shown in FIG. 1A illustrates the inner lattice structure 121, 122 of the 3-D printed lattice bone suture anchor that provides a rough, porous surface to allow for incorporation of bone growth without loss of fixation. In particular, the inner lattice structure 121, 122 may feature a cross-sectional structure 122 with spaces 121 left between, thereby providing the rough, porous surface to allow the ingrowth of a patient's own bone quality to lessen the chance of rejection and loss of strength. For example, once the screw 100 is placed within the bone, the 3-D printed lattice structure 121, 122 of the middle section 120 enables ingrowth of the bone in and around the 3-D printed lattice structure 121, 122 for improved fixation of the bone within the screw and suture anchor 100. In an embodiment, the middle section 120 may comprise a 3-D printed porous inner lattice structure 121, 122 with a rough, porous surface that improves fixation. In other embodiments, the geometry and dimensions of the inner lattice structure 121, 122 of the middle section 120 of the 3-D printed titanium porous screw may generally be dependent upon a patient's bone, joint and tendon attributes, as determined by pre-operative surgical planning with radiographs, MRI, and CT scans. Such patient specific anatomical attributes may determine the 3-D printed manufacturing of the inner lattice structure 121, 122 of the middle section 120 of the 3-D printed titanium porous screw. For example, the geometry of the inner lattice structure, including the cross-sectional structure 122 that defines the spaces 121 left between the cross-sectional structure 122 may comprise triangular, quadrilateral, pentagonal, hexagonal, heptagonal, and octagonal shapes. Of course, additional geometric shapes and other configurations are contemplated to achieve a patient-specific, 3-D printed lattice formation for placement of the screw to facilitate ingrowth of a patient's own bone quality to lessen the chance of rejection and loss of strength.

The lower section 130 shown in FIG. 1A may be a non-porous material and composed of the same material as the rest of the screw 100, such as a titanium material, for example. The screw with suture anchor 100 of FIG. 1A eliminates most of the drawbacks associated with the existing suture anchors and Biotenodesis screws that utilize Poly L Lactic Acid (PLLA) and other composites that can lead to delayed reaction and loosening of fixation. In other embodiments, the size and dimensions of each of the top 110, middle 120 and lower 130 sections of the 3-D printed titanium porous screw 100 may generally be dependent upon a patient's bone, joint and tendon attributes, as determined by pre-operative surgical planning with radiographs, MRI, and CT scans. Such patient specific anatomical attributes may determine the 3-D printed manufacturing of each of the top 110, middle 120 and lower 130 sections of the 3-D printed titanium porous screw 100.

Referring to FIG. 1B, the screw 100 may also feature a head surface 111 with a perimeter edge 115 further defining and including a loop member 116 extending along a diameter of the head surface 111. The loop member 116 provides functionality to secure a FiberWire suture to the head surface 111 of the screw 100. As aforementioned, once the screw with suture anchor 100 is placed within a bone tunnel, a number 2 FiberWire suture, for example, may be looped and secured to the loop member 116 and thereafter used to create a stitch around the bone to allow the appropriate tendon to bone insertion. For example, the free end of the suture that is not secured to the loop member 116 may then be tied to soft tissue, such as a ligament, thereby securing the soft tissue to the bone.

FIG. 1C illustrates a FiberWire suture 117 looped around the loop member 116 of the head surface 111 of the screw 100. More specifically, FIG. 1C illustrates the functionality of the loop member 116 on the head surface 111, with a FiberWire suture 117 attached to the loop member 116 of the screw 100. When placing the screw 100 into a bone tunnel, the head surface 111 of the screw 100 may comprise a recessed space 119 within the head surface 111 and underneath the loop member 116, which provides an attachment point for the FiberWire suture 117. In general, once the screw 100 with loop member 116 is placed within the bone tunnel, a number two FiberWire suture 117 may be looped underneath the loop member 116 and used to create a stitch around the bone to allow the appropriate tendon to bone insertion.

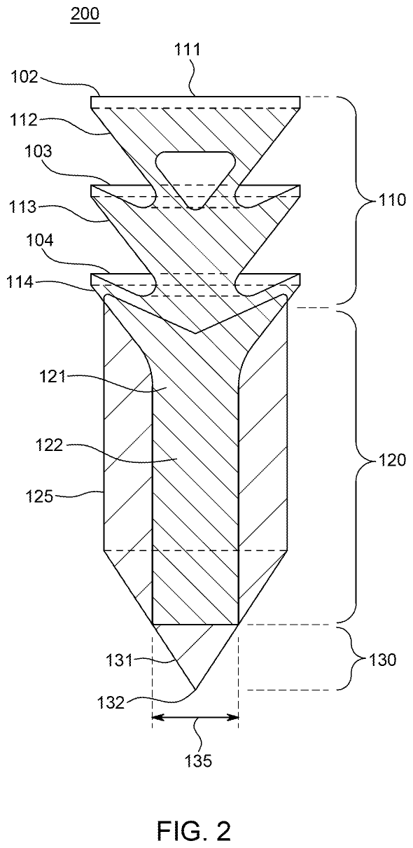

Further, FIG. 2 shows a cross-sectional view of a 3-D printed titanium porous screw with suture anchor 200, where a patient's bone, joint, and tendon attributes may determine the dimensions and parameters of the 3-D printed manufacturing process for the screw 200. The cross-sectional view of FIG. 2 includes a top section 110 with head surface 111, substantially planar ledges 102, 103, 104 extending laterally and perpendicularly to the axis of the screw 100, and step-tapered edges 112, 113, 114 respectively meeting each ledge 102, 103, 104 at an angle, which may generally be less than 90 degrees to provide a screw thread in the top section 110. The cross-sectional view of FIG. 2 further illustrates the middle section 120 with the 3-D printed inner lattice structure that provides a rough, porous surface to allow for incorporation of bone growth without loss of fixation. As shown in FIG. 2, the inner lattice structure of the middle section 120 may feature a cross-sectional structure 122 with spaces 121 left between, thereby providing the rough, porous surface to allow the ingrowth of a patient's own bone quality to lessen the chance of rejection and loss of strength. The cross-sectional view of FIG. 2 also shows an outer edge 125 of the 3-D printed lattice structure 122, 121 of the middle section 120. The cross-sectional view of FIG. 2 further illustrates a lower section 130 having a diameter 135 and an angled edge 131 leading to a point 132 for insertion into a bone tunnel. In an embodiment, the diameter 135 of the lower section 130 is approximately 1.5 millimeters. Further, the lower section 130 may be a non-porous material and composed of the same material as the rest of the screw 200, such as a titanium material, for example. The cross-sectional view of FIG. 2 illustrates the 3-D printed inner-lattice structure 121, 122, 125 of the middle section 120 that enables ingrowth of the bone in and around the 3-D printed lattice structure 121, 122 for improved fixation of the bone within the screw and suture anchor 200.

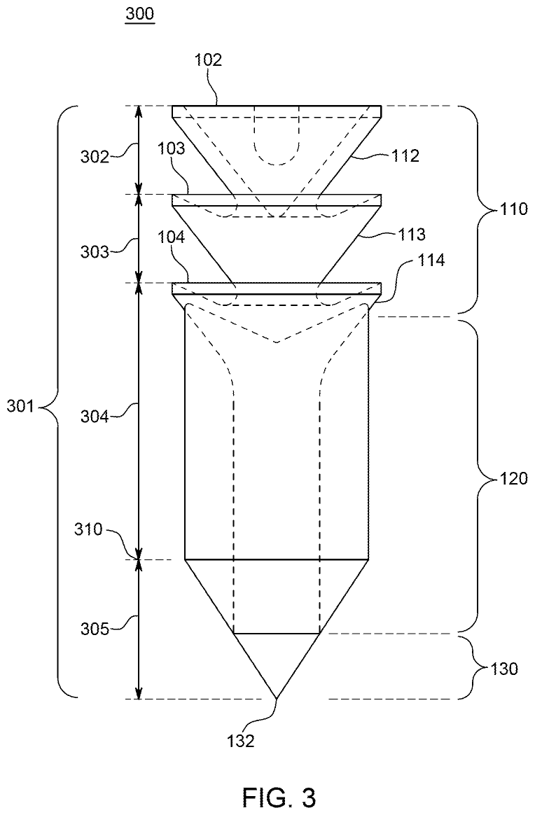

Referring to FIG. 3 showing a cross-sectional view of an embodiment of a 3-D printed titanium porous screw with suture anchor 300, the top section 110 with substantially planar ledges 102, 103, 104 extending laterally and perpendicularly to the axis of the screw 300 and step-tapered edges 112, 113, 114 increases insertion torque and provides for better bone-to-implant contact, better implant insertion, improved bone incorporation and enhanced soft tissue and bone fixation. In particular and as shown in FIG. 3, step-tapered edges 112, 113 of the top section 110 each comprise a respective height 302, 303. The height 302 of edge 112 extends between ledge 102 and ledge 103. The height 303 of edge 113 extends between ledge 103 and ledge 104. The respective heights 302, 303 of the step-tapered edges 112, 113 may be the same or approximately similar. In an embodiment, the respective heights 302, 303 of the step-tapered edges 112, 113 may alternatively be different to achieve a desired insertion torque, for example. In another embodiment, the heights 302, 303 may each be approximately 1.5 millimeters.

Further, as shown in FIG. 3, in an embodiment, the screw 300 may also feature a distance 304 between ledge 104 of tapered edge 114 and the base 310 of the middle section 120. In one embodiment, the distance 304 between ledge 104 of tapered edge 114 and the base 310 of the middle section 120 may be approximately 4.7 millimeters. In view of FIG. 3, it is understood that the distance 304 extends from the base 310 of the middle section 120 to the nearest, most adjacent ledge of the top section 110, which is ledge 104, for purposes of FIG. 3. In addition, in an additional embodiment, and as shown in FIG. 3, the screw 300 may feature a distance 305 between the base 310 of the middle section 120 and the point 132 of the lower section 130. In one embodiment, the distance 305 between the base 310 of the middle section 120 and the point 132 of the lower section 130 may be approximately 2.3 millimeters. Further, in another embodiment, the height 301 of the screw 300 may be approximately 10 millimeters. Although the screw 300 in the embodiment of FIG. 3 is illustrated with particular dimensions with respect to the heights 302, 303 of the tapered edges 112, 113, distance 304 between the ledge 104 of tapered edge 114 and base 310 of the middle section 120, distance 305 between the base 310 of the middle section 120 and the point 132 of the lower section 130, and height 301 of the screw 300, it is understood that various dimensions may be implemented for these structural features, to accommodate patient-specific attributes and anatomy, as determined by pre-operative radiographs, MRI and CT scans.

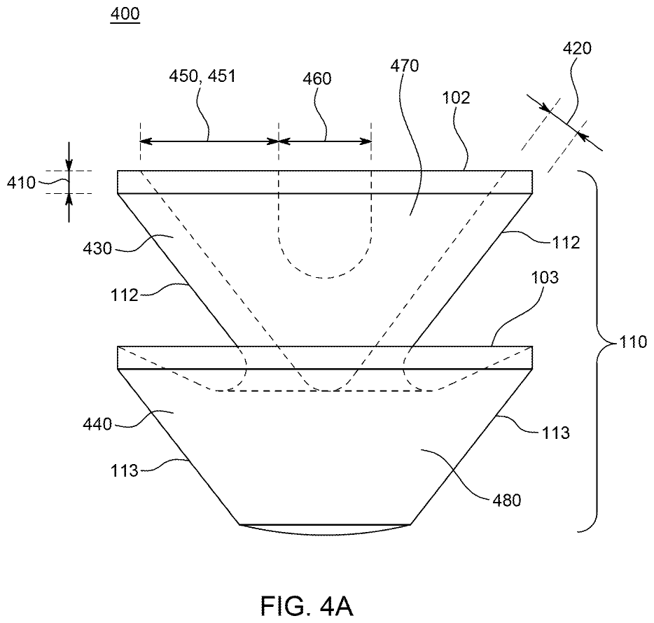

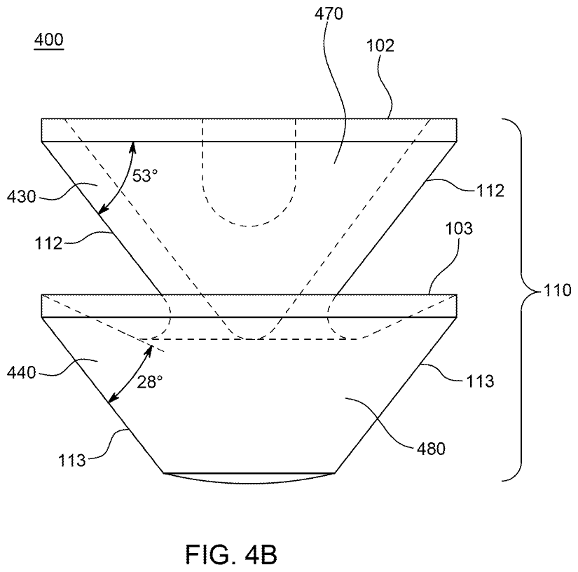

Referring to FIG. 4A showing an additional cross-sectional view of an embodiment of a 3-D printed titanium porous screw with suture anchor 400, substantially planar ledges 102, 103 and step-tapered edges 112, 113 of the top section 110 are shown. The substantially planar ledges 102, 103 each feature step-tapered edges 112, 113, respectively. The substantially planar ledges 102,103 each extend laterally and perpendicularly to the axis of the screw 400. Although not shown in FIG. 4A, it is understood that the top section 110 may also include ledge 104 and tapered edge 114 (as shown in FIG. 1, for example).

In addition, FIG. 4B illustrates the angle 430 between ledge 102 and edge 112, and the angle 440 between ledge 103 and edge 113. The angle 430 between ledge 102 and edge 112 may generally be less than 90 degrees to provide a screw thread. Likewise, the angle 440 between ledge 103 and edge 113 may generally be less than 90 degrees to provide a screw thread. As shown in FIG. 4B, in an embodiment, the angle 430 between ledge 102 and edge 112 may be approximately 53 degrees, and the angle 440 between ledge 103 and edge 113 may be approximately 53 degrees. Accordingly, the top section 110 with substantially planar ledges 102, 103 and angled, step-tapered edges 112, 113 may increase insertion torque and improve bone-to-implant contact, implant insertion, bone incorporation and soft tissue and bone fixation.

Referring again to FIG. 4A to further detail the structure of the top section 110 of an embodiment of a 3-D printed titanium porous screw 400, each of the planar ledges 102,103 and step-tapered edges 112, 113 of the top section 110 may feature a respective thickness. For example, the thickness of the planar ledge 102 may be a distance 410, as shown in FIG. 4A. In an embodiment, the thickness 410 of the planar ledge 102 may be approximately 0.2 millimeters. In addition, the thickness of the step-tapered edge 112 may be a distance 420, as shown in FIG. 4A. In an embodiment, the thickness 420 of the step-tapered edge 112 may be approximately 0.3 millimeters. In another embodiment, the respective distances 410, 420 of the planar ledge 102 and step-tapered edge 112 may be the same with respect to the corresponding planar ledge 103 and tapered edge 113. In addition, the top section 110 of the screw 400 shown in FIG. 4A may include hollow interiors 470, 480 disposed respectively within the planar ledge 102 and tapered edge 112, and the planar ledge 103 and tapered edge 113.

In addition, and with respect to FIG. 4A in conjunction with FIGS. 1B and 1C, the loop member 116 may feature a width 460, as shown in FIG. 4A. The loop member 116 provides functionality to secure a FiberWire suture 117 to the head surface 111 of the screw 400. In an embodiment, the loop member 116 may feature a width 460 of approximately 0.8 millimeters. Once the screw with suture anchor 400 is placed, a number 2 FiberWire suture, for example, may be looped and secured to the loop member 116 and thereafter used to create a stitch around the bone to allow the appropriate tendon to bone insertion.

Further, and with respect to FIG. 4A in conjunction with FIGS. 1B and 1C illustrating the recessed space 119 within the head surface 111 and planar ledge 102 of the screw 100, 400, the recessed space 119 may be accessed through the opening 451 in the head surface 111 and planar ledge 102 of the screw 100, 400. The opening 451 may comprise a distance 450. In an embodiment, the distance 450 may be approximately 1.12 millimeters. The opening 451 with distance 450 may be symmetrical with respect to each side of the loop member 116 that includes a width 460, so that a FiberWire suture 117 may be looped and secured around the loop member 116. More specifically, a FiberWire suture 117 be inserted through the opening 451 on a side of the loop member 116, looped around the loop member 116, and pulled through the other corresponding opening 451 in the head surface 111 and planar ledge 102 of the screw 100, 400.

Although the screw with suture anchor 400 in the embodiments of FIGS. 4A and 4B is illustrated with particular dimensions with respect to the thickness 410 of the of the planar ledges 102, 103, the thickness 420 of the tapered edges 112, 113, the width 460 of the loop member 116, the distance 450 of the opening 451 into the recessed space 119 through the head surface 111 and planar ledge 102, the angle 430 between ledge 102 and edge 112, and the angle 440 between ledge 103 and edge 113, it is understood that various dimensions and angles may be implemented for these structural parameters, to accommodate patient-specific attributes and anatomy, as determined by pre-operative radiographs, MRI and CT scans.

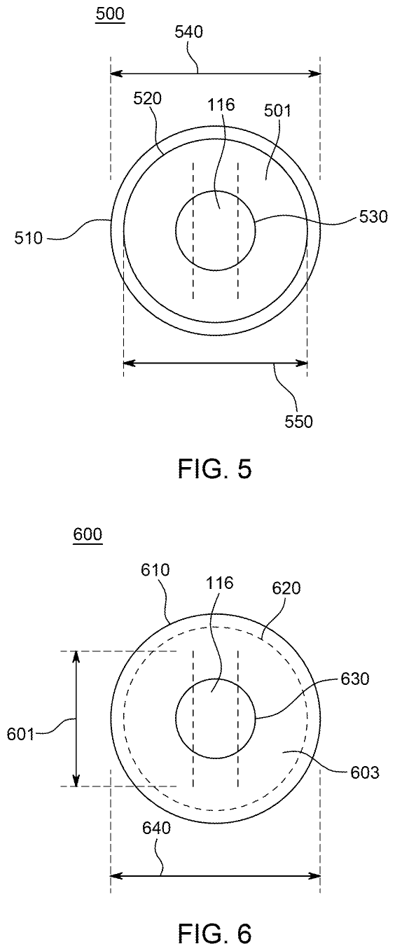

Referring to FIG. 5 showing an additional cross-sectional view of an embodiment of a 3-D printed titanium porous screw with suture anchor 500, an axial view of the diameter 540 of the outer titanium structure 510 of the screw 500 is shown, along with the diameter 550 of the inner lattice structure 520, which allows for improved bone ingrowth. In particular, the axial view of FIG. 5 illustrates a diameter 540 of the outer titanium structure 510 of the screw 500, which may be approximately 3.5 millimeters. In addition, FIG. 5 shows a diameter 550 of the inner lattice structure 520, which may be approximately 3 millimeters. The cross-sectional view provided by FIG. 5 also shows the circular perimeter 530 of the lower section 130 of FIG. 1A. Between the circular perimeter 530 of the lower section 130 and the perimeter 520 of the inner lattice structure is the 3-D printed porous inner lattice structure 501.

Referring to FIG. 6, showing an additional cross-sectional view of an embodiment of a 3-D printed titanium screw with suture anchor 600, an axial view of the diameter 640 of the outer titanium structure 610 of the screw 600 is shown, along with the length 601 of the loop member 116 (shown in FIGS. 1B and 1C). In particular, the axial view of FIG. 6 illustrates a diameter 640 of the outer titanium structure 610 of the screw 600, which may be approximately 3.5 millimeters. In addition, FIG. 6 shows a perimeter 620 of the inner lattice structure 603. The cross-sectional view provided by FIG. 6 also shows the circular perimeter 630 of the lower section 130 of FIG. 1A. Between the circular perimeter 630 of the lower section 130 and the perimeter 620 of the inner lattice structure is the 3-D printed porous inner lattice structure 603.

In addition, and with respect to FIG. 6 in conjunction with FIGS. 1B and 1C, the loop member 116 may feature a length 601, as shown in FIG. 6. The loop member 116 provides functionality to secure a FiberWire suture 117 to the head surface 111 of the screw 600. In an embodiment, the loop member 116 may feature a length 601 of approximately 2.1 millimeters. Once the screw with suture anchor 600 is placed, a number 2 FiberWire suture, for example, may be looped and secured to the loop member 116 and thereafter used to create a stitch around the bone to allow the appropriate tendon to bone insertion.

Having thus described the presently preferred embodiments in detail, it is to be appreciated and will be apparent to those skilled in the art that many physical changes, only a few of which are exemplified in the detailed description of the invention, could be made without altering the inventive concepts and principles embodied therein. It is also to be appreciated that numerous embodiments incorporating only part of the preferred embodiment are possible which do not alter, with respect to those parts, the inventive concepts and principles embodied therein. The present embodiments and optional configurations are therefore to be considered in all respects as exemplary and/or illustrative and not restrictive, of the scope of the invention being indicated by the appended claims rather than by the foregoing description, and all alternate embodiments and changes to this embodiment which come within the meaning and range of equivalency of said claims are therefore to be embraced therein.

What is claimed is:

1. A surgical screw comprising:
a top section having:
a head surface comprising a loop member for attachment of a suture to the screw;
a plurality of substantially planar ledges each having a step-tapered edge; and
an angle disposed between each of the substantially planar ledges and the respective step-tapered edges;
a middle section having a 3-D printed porous inner lattice structure; and
a lower section including a point configured for insertion into a bone tunnel,
wherein the middle section is disposed between the top section and the lower section; and
wherein the 3-D printed porous inner lattice structure is dependent upon a patient's bone, joint, and tendon attributes as determined by pre-operative surgical planning with radiographs, MRI, and CT scans.

2. The surgical screw of claim 1, wherein each of the top, middle and lower sections is comprised of titanium.

3. The surgical screw of claim 1, wherein the 3-D printed porous inner lattice structure of the middle section is comprised of a cross-sectional structure with a plurality of spaces between the cross-sectional structure.

4. The surgical screw of claim 1, wherein a recessed space is disposed within the top section and underneath the loop member,
wherein the recessed space is accessible through an opening in the head surface for attachment of the suture to the loop member of the screw.

5. The surgical screw of claim 1, wherein the top section comprises at least three substantially planar ledges each having a respective step-tapered edge.

6. The surgical screw of claim 1, wherein the angle disposed between each of the substantially planar ledges and the respective step-tapered edges is less than 90 degrees.

7. The surgical screw of claim 1, wherein the angle disposed between each of the substantially planar ledges and the respective step-tapered edges is approximately 53 degrees.

8. The surgical screw of claim 1, wherein each step-tapered edge includes a height.

9. The surgical screw of claim 8, wherein at least one height of the step-tapered edges is approximately 1.5 millimeters.

10. The surgical screw of claim 1, wherein the middle section further comprises a base and a distance between the base and a nearest, most adjacent ledge of the top section, wherein the distance between the base and the nearest, most adjacent ledge of the top section is approximately 4.7 millimeters.

11. The surgical screw of claim 1, wherein the middle section further comprises a base and a distance between the base and the point of the lower section is approximately 2.3 millimeters.

12. The surgical screw of claim 1, wherein a height of the screw is approximately 10 millimeters.

13. The surgical screw of claim 1, wherein a diameter of the 3-D printed porous inner lattice structure is approximately 3 millimeters.

14. A surgical screw comprising:
a top section having a head surface comprising a loop member for attachment of a suture to the screw;
a middle section having a 3-D printed porous inner lattice structure; and
a lower section including a point configured for insertion into a bone tunnel,
wherein the middle section is disposed between the top section and the lower section; and
wherein the 3-D printed porous inner lattice structure is dependent upon a patient's bone, joint, and tendon attributes as determined by pre-operative surgical planning with radiographs, MRI, and CT scans.

15. The surgical screw of claim 14, wherein the screw is comprised of titanium.

16. The surgical screw of claim 14, wherein the 3-D printed porous inner lattice structure of the middle section is comprised of a cross-sectional structure with a plurality of spaces between the cross-sectional structure.

17. The surgical screw of claim 14, wherein a recessed space is disposed within the top section and underneath the loop member,
wherein the recessed space is accessible through an opening in the head surface for attachment of the suture to the loop member of the screw.

18. The surgical screw of claim 14, wherein the top section comprises:
a plurality of substantially planar ledges each having a step-tapered edge; and
an angle disposed between each of the substantially planar ledges and the respective step-tapered edges,
wherein the angle disposed between each of the substantially planar ledges and the respective step-tapered edges is less than 90 degrees.

19. The surgical screw of claim 14, wherein a diameter of the 3-D printed porous inner lattice structure is approximately 3 millimeters.

20. A surgical screw comprising:
a top section having a head surface comprising a loop member for attachment of a suture to the screw; and
a middle section having a 3-D printed porous inner lattice structure,
wherein the 3-D printed porous inner lattice structure of the middle section is comprised of a cross-sectional structure with a plurality of spaces between the cross-sectional structure;
wherein a diameter of the 3-D printed porous inner lattice structure is approximately 3 millimeters; and
wherein the 3-D printed porous inner lattice structure is dependent upon a patient's bone, joint and tendon attributes as determined by pre-operative surgical planning with radiographs, MRI, and CT scans.
